(19) United States
(12) Patent Application Publication
(10) Pub. No.: US 2026/0351376 A1
(43) Pub. Date: Nov. 5, 2026

(54) MACHINE LEARNING SYSTEM AND METHOD USING CLINICAL DATA TO PREDICT RESPONSE TO INTRALESIONAL STEROIDS IN KELOID PATIENTS

(71) Applicant: The Florida State University Research Foundation, Inc., Tallahassee, FL (US)

(72) Inventors: Akash Gunjan, Tallahassee, FL (US); Nina Zamani, Tallahassee, FL (US); Parand Akbari, Tallahassee, FL (US); Masoud Zamani, Tallahassee, FL (US)

(21) Appl. No.: 19/666,911

(22) Filed: May 4, 2026

Related U.S. Application Data
(60) Provisional application No. 63/799,013, filed on May 2, 2025.

(51) Int. Cl. G16H 20/10; G16H 80/00

(52) U.S. Cl. CPC G16H 20/10; G16H 80/00

(57) ABSTRACT

An exemplary system and method that employ trained AI models that can provide, e.g., in pre-treatment predictions, for the response of keloid patients to steroid therapy, enabling a more personalized and effective treatment for keloid patients. The trained AI model may be employed with a webapp-based tool or other software portal, for clinicians to predict steroid response for keloids patients at the point of care on their mobile devices. Clinicians can input keloid parameters and patient demographic data to obtain a prediction regarding the use of steroids for keloid therapy in individual patients. Although the models were developed using a large dataset from keloid patients, similar approaches with less data can be used to yield useful ML models for predicting response to steroid therapy in other conditions as well.

### RELATED APPLICATION

This application claims priority to, and the benefit of, U.S. Provisional Patent Application No. 63/799,013, filed May 2, 2025, entitled “Leveraging Machine Learning and Clinical Data to Predict Response to Intralesional Steroids in Keloid Patients,” which is incorporated by reference herein in its entirety.

### BACKGROUND

Keloid disorders are common but non-lethal fibroproliferative skin tumors characterized by excessive fibroblast proliferation and overproduction of type I collagen.

Although keloid lesions are widely treated using intralesional corticosteroid injections, many patients do not benefit, while others get worse. Corticosteroids (or simply steroids) like triamcinolone acetonide (TA) are a widely used first-line therapy for keloid disorder, yet many patients show limited or no response, while a significant number see a worsening of their keloids. Steroid resistance is observed in the treatment of other conditions as well, including but not limited to pediatric nephrotic syndrome, asthma, and inflammatory bowel diseases. Given the variability of outcomes following steroid treatment, there is a pressing need for tools that can predict patients’ steroid response prior to initiating therapy.

There are currently no means to predict response to intralesional corticosteroid treatment for keloids.

There is a benefit to improving the treatment of keloid disorder and other disorders to which steroid resistance affects such treatment.

### SUMMARY

An exemplary system and method are disclosed that employ trained AI models that can provide, e.g., in pre-treatment predictions, for the response of keloid patients to steroid therapy, enabling a more personalized and effective treatment for keloid patients. The trained AI model may be employed with a webapp-based tool or other software portal, for clinicians to predict steroid response for keloids patients at the point of care on their mobile devices. Clinicians can input keloid parameters and patient demographic data to obtain a prediction regarding the use of steroids for keloid therapy in individual patients. Although the models were developed using a large dataset from keloid patients, similar approaches with less data can be used to yield useful ML models for predicting response to steroid therapy in other conditions as well.

The machine learning (ML) models are the first tools that can predict a keloid patient's response to steroid therapy with 95% accuracy based solely on basic clinical data regarding keloid parameters such as shape, and patient demographics, e.g., sex and age. A study was conducted that evaluated 9 trained AI models; it was observed that all 9 ML models performed with accuracy >90%, with Neural Network (NN) and Random Forest (RF) providing accuracy of ~95%, which was beneficial as NN is also straightforward for scalability. Using this algorithm, clinicians can quickly predict the likely response of keloid patients to steroid therapy and administer it only to patients who are likely to respond, while avoiding unnecessary and potentially harmful treatments for patients likely to be resistant to steroid therapy.

Any patient with keloids or a disorder where steroids are currently used for therapy can benefit from the exemplary system and method. Physicians (especially dermatologists, immunologists, rheumatologists) who prescribe steroids, pharmacies that dispense steroids, as well as diagnostic laboratories may utilize the algorithm or portal, for predicting response to steroids.

In some aspects, the techniques described herein relate to a system including: a processor; and a memory having instructions stored thereon, wherein execution of the instructions by the processor, causes the processor to: receive input (e.g., from a web interface) including three or more of: gender for a patient, shape of a keloid on the patient, 10-year growth history of the keloid on the patient, 5-year growth history of the keloid on the patient, 2-year growth history of the keloid on the patient, 1-year growth history of the keloid on the patient, location of the keloid on the patient, current age of the patient, and demographic information of the patient, determine, via a trained AI model, a predicted response of a patient to a steroid dose for the treatment of said keloid; and output the determined predicted response, wherein the determined predicted response is employed to treatment of the patient by steroid to treat the keloid.

In some aspects, the techniques described herein relate to a system, wherein the input includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, wherein the trained AI model was trained using: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient.

In some aspects, the techniques described herein relate to a system, wherein the input includes: wherein the input includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, current age of the patient, and demographic information of the patient, wherein the trained AI model was trained using: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, the location of the keloid on the patient, the current age of the patient, and demographic information of the patient,

In some aspects, the techniques described herein relate to a system, wherein the trained AI model is a neural network.

In some aspects, the techniques described herein relate to a system, wherein the trained AI model uses at least one of random forest, SVS, XGBoost, logistic regression, gradient boosting, Catboost, bagging, or adaboost.

In some aspects, the techniques described herein relate to a system, wherein the input is provided via a web interface from a web portal.

In some aspects, the techniques described herein relate to a system, wherein the output is provided via a web interface from a web portal.

In some aspects, the techniques described herein relate to a system, wherein the trained AI model was trained without at least one of patient race, keloid trigger information, patient age, keloid onset age, and patient demographic information.

In some aspects, the techniques described herein relate to a method including: receiving, by a processor, input (e.g., from a web interface) including three or more of: gender for a patient, shape of a keloid on the patient, 10-year growth history of the keloid on the patient, 5-year growth history of the keloid on the patient, 2-year growth history of the keloid on the patient, 1-year growth history of the keloid on the patient, location of the keloid on the patient, current age of the patient, and demographic information of the patient, determining, by the processor, via a trained AI model, a predicted response of a patient to a steroid dose for the treatment of said keloid; and outputting, by the processor, the determined predicted response, wherein the determined predicted response is employed to treatment of the patient by steroid to treat the keloid.

In some aspects, the techniques described herein relate to a method, wherein the input includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, wherein the trained AI model was trained using: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient.

In some aspects, the techniques described herein relate to a method, wherein the input includes: wherein the input includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, current age of the patient, and demographic information of the patient, wherein the trained AI model was trained using: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, the location of the keloid on the patient, the current age of the patient, and demographic information of the patient,

In some aspects, the techniques described herein relate to a method, wherein the trained AI model is a neural network.

In some aspects, the techniques described herein relate to a method, wherein the trained AI model uses at least one of random forest, SVS, XGBoost, logistic regression, gradient boosting, Catboost, bagging, or adaboost.

In some aspects, the techniques described herein relate to a method, wherein the input is provided via a web interface from a web portal.

In some aspects, the techniques described herein relate to a method, wherein the output is provided via a web interface from a web portal.

In some aspects, the techniques described herein relate to a method, wherein the trained AI model was trained without at least one of patient race, keloid trigger information, patient age, keloid onset age, and patient demographic information.

In some aspects, the techniques described herein relate to a non-transitory computer-readable medium having instructions stored thereon, wherein execution of the instructions by a processor causes the processor to: receive input (e.g., from a web interface) including three or more of: gender for a patient, shape of a keloid on the patient, 10-year growth history of the keloid on the patient, 5-year growth history of the keloid on the patient, 2-year growth history of the keloid on the patient, 1-year growth history of the keloid on the patient, location of the keloid on the patient, current age of the patient, and demographic information of the patient, determine, via a trained AI model, a predicted response of a patient to a steroid dose for the treatment of said keloid; and output the determined predicted response, wherein the determined predicted response is employed to treatment of the patient by steroid to treat the keloid.

In some aspects, the techniques described herein relate to a non-transitory computer-readable medium, wherein the input includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, wherein the trained AI model was trained using: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient.

In some aspects, the techniques described herein relate to a non-transitory computer-readable medium, wherein the input includes: wherein the input includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, current age of the patient, and demographic information of the patient, wherein the trained AI model was trained using: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, the location of the keloid on the patient, the current age of the patient, and demographic information of the patient,

In some aspects, the techniques described herein relate to a non-transitory computer-readable medium, wherein the trained AI model is a neural network.

In some aspects, the techniques described herein relate to a non-transitory computer-readable medium, wherein the trained AI model uses at least one of random forest, SVS, XGBoost, logistic regression, gradient boosting, Catboost, bagging, or adaboost.

In some aspects, the techniques described herein relate to a non-transitory computer-readable medium, wherein the input is provided via a web interface from a web portal, and wherein the output is provided via a web interface from a web portal.

### BRIEF DESCRIPTION OF DRAWINGS

FIGS. 1A and 1B show an example system that employs a trained AI model to provide an estimated/predicted response of keloid patients to steroid therapy in accordance with an illustrative embodiment.

FIG. 1C shows an example input and output of a web portal that employed the trained AI model of FIGS. 1A or 1B to provide an estimated/predicted response of keloid patients to steroid therapy in accordance with an illustrative embodiment.

FIGS. 2A and 2B show examples of permutation and SHAP important analysis determined in a study that provided example inputs for the trained AI system for inference.

FIG. 3 shows accuracy results for a set of AI models developed in a study.

FIG. 4A shows features employed in the training of the AI models.

FIG. 4B shows training and development process employed in the training of the AI models in the study.

FIG. 5, subpanels A – D show SHAP parameters showing the relative importance of different features, as well as interactions between specific features in the model. FIG. 5, subpanel A, shows Global SHAP bar plot illustrating the average importance of feature groups in determining the model’s prediction of steroid therapy sensitivity. Patient gender and the morphology of keloid lesions exhibit the highest mean SHAP values, highlighting their strong influence on treatment outcomes. FIG. 5, subpanel B, shows SHAP summary plot showing the effect of individual features on the predicted response to steroid therapy. Positive SHAP values indicate sensitivity to treatment, while negative values suggest resistance. The color gradient represents feature values, where red indicates high values and blue indicates low values. Notably, patient gender and the morphology of keloid lesions exhibit a range of effects depending on the specific patient. FIG. 5, subpanel C, shows SHAP Interaction Plot revealing the interaction between age and gender in predicting response to steroid therapy. Positive SHAP interaction values indicate that the interaction drives the prediction toward sensitivity, while negative values suggest resistance. This plot suggests that younger female patients are more likely to be sensitive to treatment, with a diminishing effect as age increases. FIG. 5, subpanel D, shows Group-level permutation importance, highlighting the standalone effect of feature groups on model accuracy. Patient gender and the morphology of keloid lesions remain the most critical predictors, reinforcing findings from the SHAP analysis, though age demonstrates less direct influence.

FIG. 6, subpanels A – D show the performance of the trained AI models. FIG. 6, subpanel A, shows a heatmap of performance metrics for various ML models, sorted by accuracy. FIG. 6, subpanel B, show spider plot illustrating the performance metrics for various ML models. FIG. 6, subpanel C, shows AUC values for the different ML models. FIG. 6, subpanel D, shows the effect of training set size on F0.5 scores across different ML models. Increasing the proportion of data significantly enhances performance, underscoring the positive impact of larger training sets on model effectiveness.

FIG. 7 shows hyperparameter optimization details for each of the trained AI models of FIG. 3.

FIG. 8, subpanels A – I, shows details on the dataset employed for the training of the AI models of FIG. 3. FIG. 8, subpanel A, shows the distribution of gender. FIG. 8, subpanel B, shows the distribution of race or nationality. FIG. 8, subpanel C, shows the distribution of the age of the patients. FIG. 8, subpanel D, shows the distribution of the age of keloid onset. FIG. 8, subpanel E, shows the distribution of a number of prior steroid injections. FIG. 8, subpanel F shows a total number of steroid injections a patient had received for their keloids. FIG. 8, subpanel G, shows the distribution of keloid triggers. FIG. 8, subpanel H, shows the distribution of keloid shape. FIG. 8, subpanel I shows example images of keloid morphologies (e.g., nodular, linear, flat, massive, superficially spreading, and pedunculated).

### DETAILED DESCRIPTION

Some references, which may include various patents, patent applications, and publications, are cited in a reference list and discussed in the disclosure provided herein. The citation and/or discussion of such references is provided merely to clarify the description of the disclosed technology and is not an admission that any such reference is “prior art” to any aspects of the disclosed technology described herein. In terms of notation, “[n]” corresponds to the nth reference in the list. For example, [1] refers to the first reference in the list. All references cited and discussed in this specification are incorporated herein by reference in their entirety and to the same extent as if each reference were individually incorporated by reference.

### Example System and Method

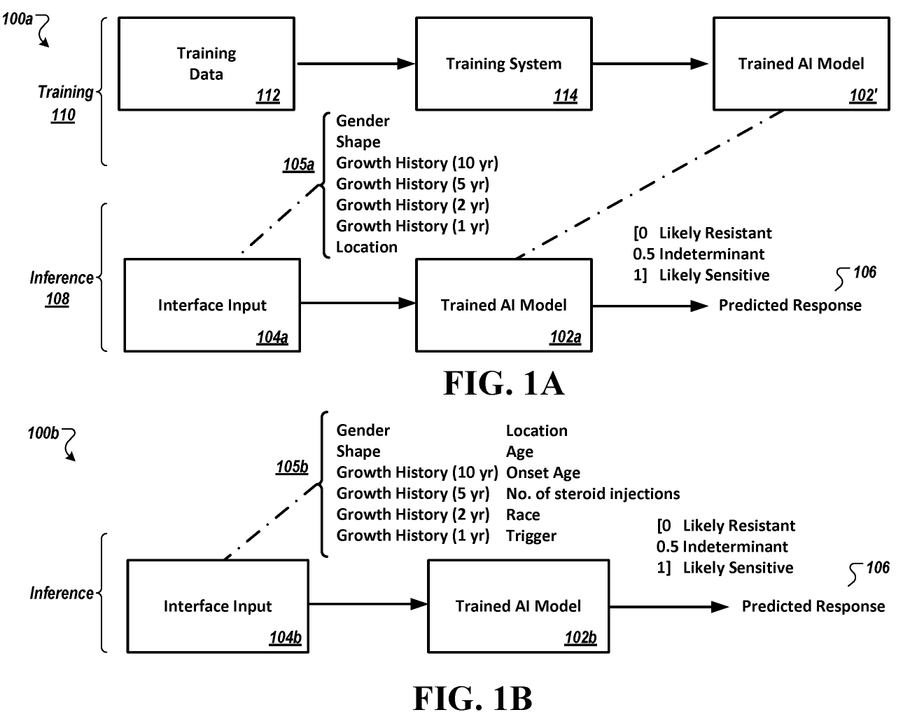

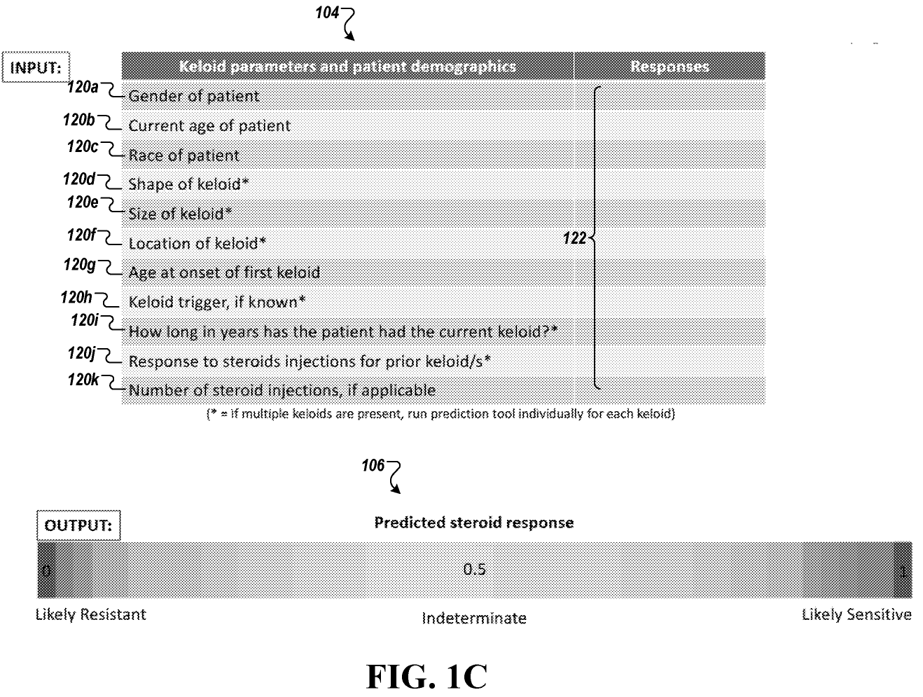

FIGS. 1A and 1B show an example system and Method 100 (shown as 100a and 100b) that employs a trained AI model 102 (shown as 102a, 102b) to provide an estimated/predicted response of keloid patients 106 to steroid therapy in accordance with an illustrative embodiment.

In FIG. 1A, during inference (e.g., runtime classifier operation), the input 104 (shown as 104a) includes data associated with a keloid patient including gender, shape, growth history (10 yr), growth history (5 yr), growth history (2 yr), growth history (1 yr), and keloid location.

In FIG. 1B, during inference (e.g., runtime classifier operation), the input 104 (shown as 104b) includes data associated with a keloid patient including gender, shape, growth history (10 yr), growth history (5 yr), growth history (2 yr), growth history (1 yr), and keloid location and additionally includes location, age, onset age, number of prior steroid injections, race, and trigger.

Example Web Portal. FIG. 1C shows an example input 104 and output 106 for a web portal that employed the trained AI model of FIGS. 1A or 1B to provide an estimated/predicted response of keloid patients to steroid therapy in accordance with an illustrative embodiment. In the example shown in FIG. 1C, the input of the web portal may include inputs 104, including the gender for a patient (120a), the current age of the patient (120b), the race of the patient (120c), the shape of a keloid on the patient (120d), the current size of the keloid (120e), the location of the keloid (120f), the keloid trigger (120g) if known, the age at which the first keloid had onset (120h), how long in years has the patient had the current keloid (120i), prior response to steroid injections for prior keloid (120j), and a number of steroid injections if applicable (120k). The number of years to which the patient had the current keloid may be used to determine a 10-year growth history of the keloid on the patient, a 5-year growth history of the keloid on the patient, a 2-year growth history of the keloid on the patient, a 1-year growth history of the keloid on the patient. The interface includes a field 122 for data to be provided. In some embodiments, the input may be via a file to which the data may be extracted. In some embodiments, the input may be provided through an APP, e.g., executing on a mobile device.

An example output response 106 may be a score, e.g., having a value between 0and 1where is 0is high likelihood that the patient will be steroid resistant and 1 is a high likelihood that the patient will be sensitive to steroid treatment. The score may be presented in color, text, and/or indicator, e.g., associated likelihood or recommendation that steroid treatment may be applied. Any patient with keloids or a disorder where steroids are currently used for therapy can benefit from the exemplary system and method. Physicians (especially dermatologists, immunologists, rheumatologists) who prescribe steroids, pharmacies that dispense steroids, as well as diagnostic laboratories may utilize the algorithm or portal, for predicting response to steroids.

Despite the widespread use ofs steroids (e.g., ILTA) for keloids, patient response is inconsistent, with only about two-thirds of the patients benefiting to different extents, while a significant fraction experiences a worsening of symptoms. The exemplary AI system can provide personalized medicine, enabling predictions of treatment outcomes using analysis from complex clinical datasets.

Example Method. In the example shown in FIG. 1A, the trained AI model 102a is configured to receive input 104a (e.g., from a web interface) comprising three or more of: the gender for a patient, the shape of a keloid on the patient, the 10-year growth history of the keloid on the patient, the 5-year growth history of the keloid on the patient, the 2-year growth history of the keloid on the patient, the 1-year growth history of the keloid on the patient, the location of the keloid on the patient, the current age of the patient, the demographic information of the patient, or a combination thereof. The input 104a is derived from the permutation importance results presented herein.

Where information is not available, it may be provided with a null or 0 value. The trained AI model 102a is configured to determine the predicted response 106 of the patient to a steroid dose for the treatment of said keloid. The trained AI model 102a is configured to output the determined predicted response 106, wherein the determined predicted response 106 is employed to treatment of the patient by steroid to treat the keloid.

In an example, a clinician or doctor of the doctor may employ the trained AI model 102, e.g., via a user interface via a web portal, to predict steroid response in keloid patients. Clinicians, the patient, nurse, or caretaker, could provide input of the relevant keloid parameters and patient demographic information to obtain a prediction on the potential steroid response for individual keloid patients to guide their clinical decision making. The input 104a is derived from the importance analysis results presented herein.

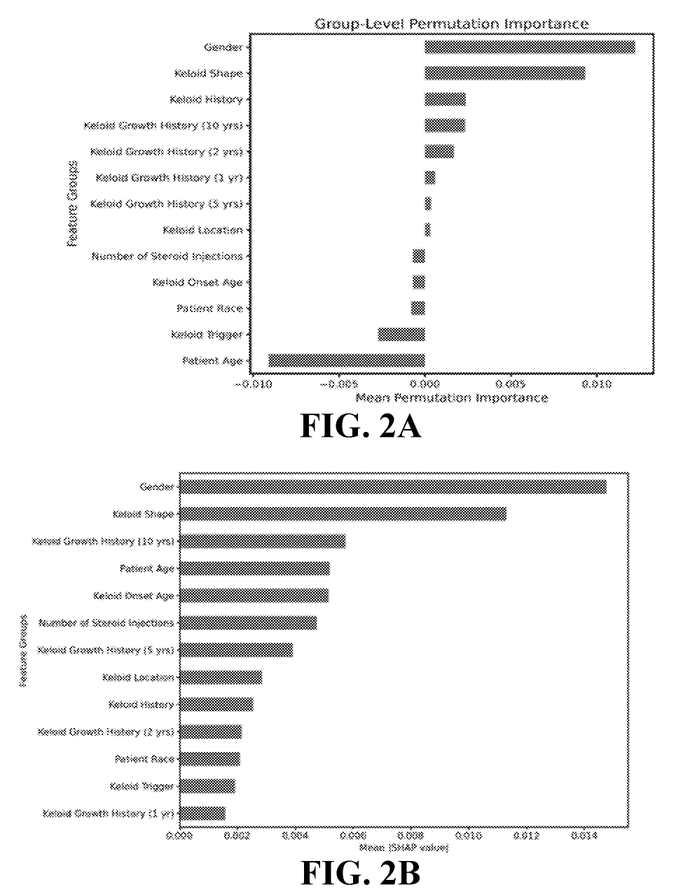

FIGS. 2A and 2B show examples of permutation and SHAP important analysis determined in a study that provided example inputs for the trained AI system for inference.

SIn some embodiments, the input 104a includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, and the location of the keloid on the patient, wherein the trained AI model was trained using that same data in the training data set; FIG. 2A shows permutation importance analysis that informs the selection of the input 104a.

In some embodiments, the input 104b (FIG. 1B) includes three or more of: the gender for the patient, the shape of a keloid on the patient, the 10-year growth history of the keloid on the patient, the 5-year growth history of the keloid on the patient, the 2-year growth history of the keloid on the patient, the 1-year growth history of the keloid on the patient, the location of the keloid on the patient, current age of the patient, and demographic information of the patient, where the trained AI model was trained using the same information, among others; FIG. 2B shows SHAP importance analysis that informs the selection of the input 104b.

The inputs (e.g., 104a, 104b) have been experimentally determined to be clinically or statistically relevant for the determination of an estimated/predicted response of keloid patients to steroid therapy in accordance with an illustrative embodiment.

In some embodiments, the trained AI model is a neural network.

In some embodiments, the trained AI model uses at least one of random forest, SVS, XGBoost, logistic regression, gradient boosting, Catboost, bagging, or adaboost.

In some embodiments, the trained AI model was trained without at least one of patient race, keloid trigger information, patient age, keloid onset age, and patient demographic information.

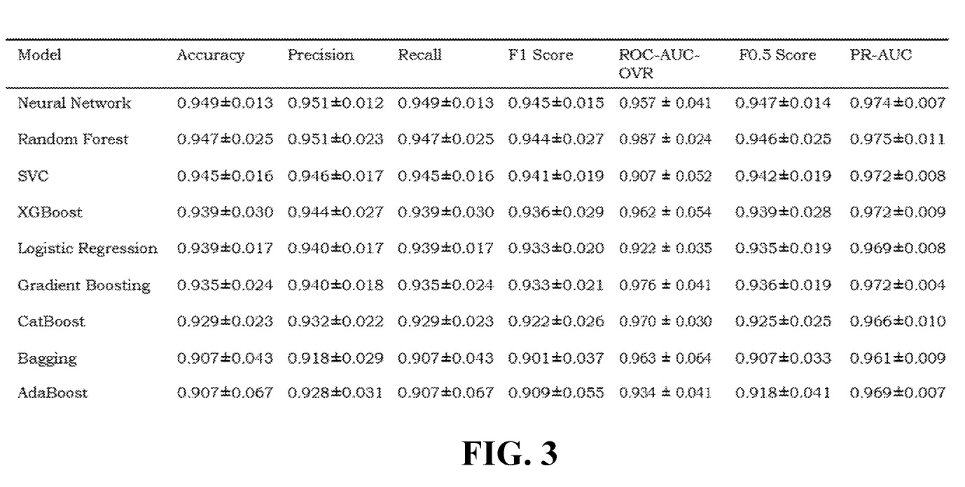

Training. In the example shown in FIG. 1A, the training 110 may include applying training data 112 to a training system 114 to generate the trained AI model 102 (shown as 102’)., which can be used as 102a, 102b. In some embodiments, the training (e.g., 110) is conducted according to the study described herein. Example of models employed in a training systems is provided in FIG. 3. FIG. 3 shows accuracy results for a set of AI models developed in the study described herein, including for neural network and random forest, SVS, XGBoost, logistic regression, gradient boosting, Catboost, bagging, or adaboost.

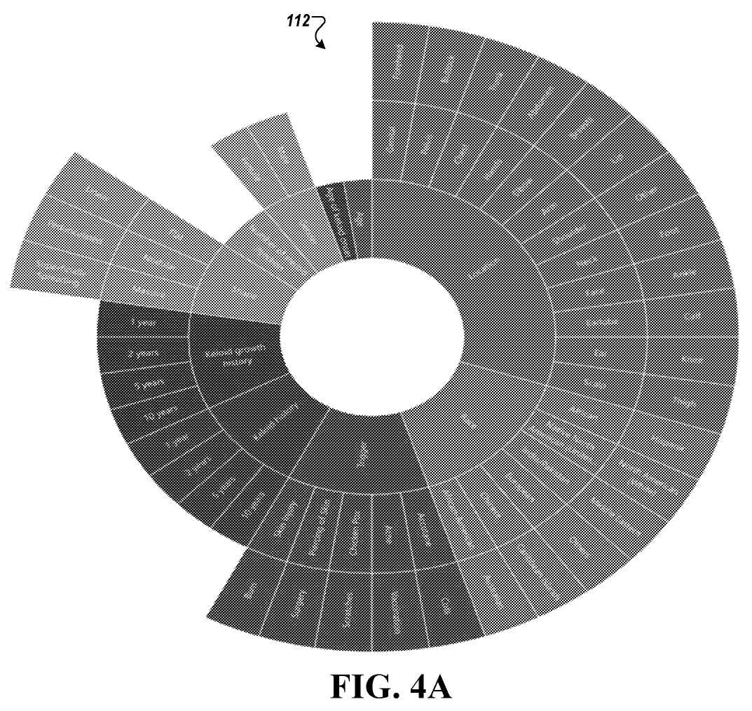

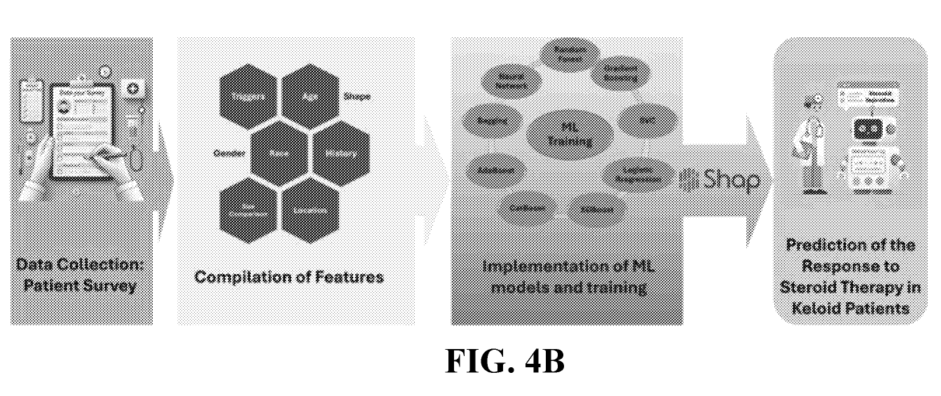

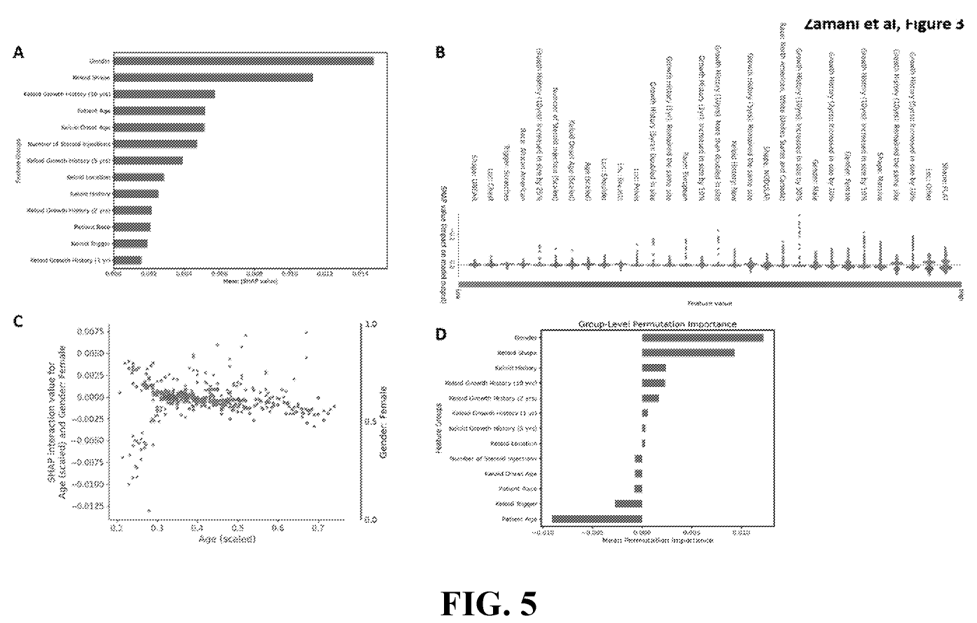

FIG. 4A shows an example set of data employed in the training data set. The training data set may be applied to a training operation and, through permutation importance analysis, important analysis, or SHAP analysis, be trained using the provided features determined to be relevant.

### Experimental Results and Additional Examples

A study was conducted to develop and evaluate a machine learning algorithm for predicting the response to intralesional steroids based on self-reported data from a large survey of 1,940 keloid patients, of whom 940 patients (290 male, 650 female) provided data on steroid treatment. Multiple ML models, including Neural Networks (NN) and Random Forest (RF), were trained using a portion of the patient survey data (the “training” set). The performance of different models was evaluated using data from another portion of the patient survey data (the “test” set) to determine predictive accuracy for steroid response in patients. Robust performance of multiple models was observed on the test dataset, with the MM and RF models resulting in ~95% predictive accuracy on our test dataset.

NN and RF-based ML models using basic keloid patient data can be used to predict response to intralesional steroids in keloid patients accurately using a web-based user interface. Similar ML models may be useful in clinical decision-making regarding the use of steroid therapy for additional conditions.

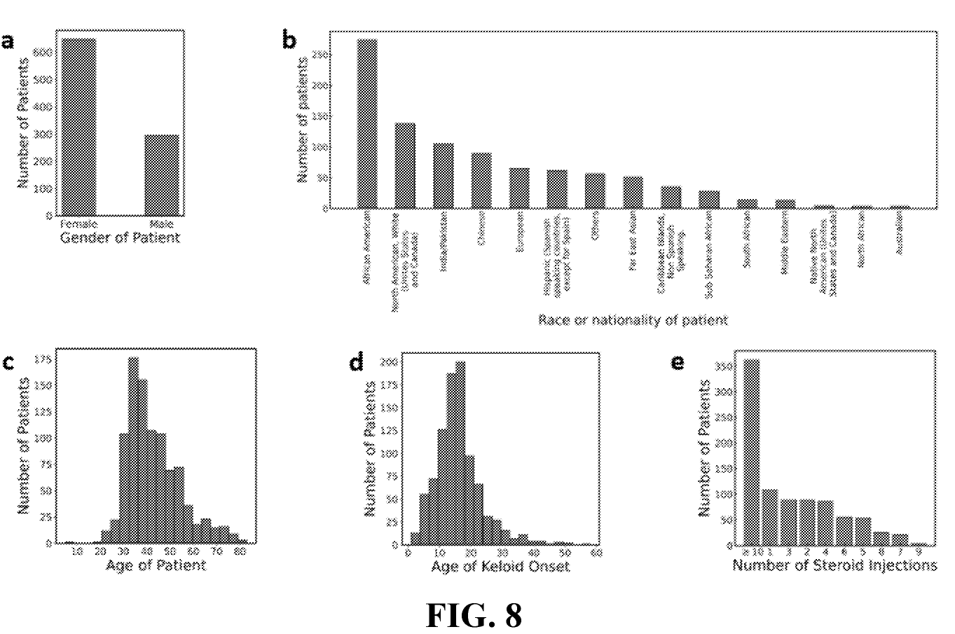

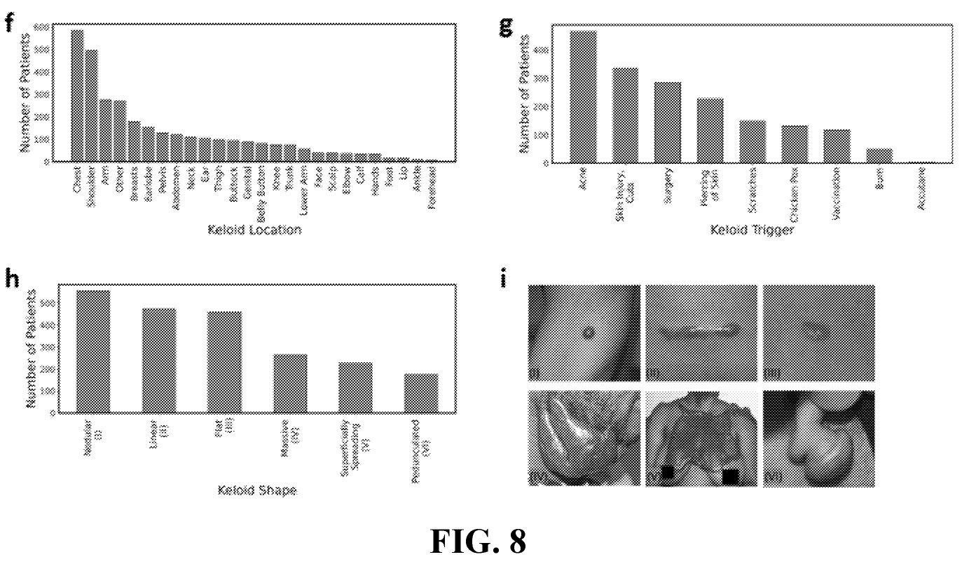

FIG. 4B shows a development and training workflow employed in the study. The ML framework was applied on a self-reported online keloid patient survey data that was published previously [6], and has been updated here in FIGS. 8. The dataset included a total of 1,940 keloid patients from whom 940 patients (290 male, 650 female) provided sufficient information on their steroid treatment to be included in the analysis. The dataset included demographic details, keloid parameters and history, treatment responses, and triggering factors. Further description of the data set is provided in [18’].

FIG. 4A shows a summary of the features employed in the study. After preprocessing, numerical features were standardized, categorical variables were one-hot encoded, and class imbalance was addressed using SMOTE-ENN. The study applied multiple ML models, including RF, Gradient Boosting (GB), XGBoost, NN, and Support Vector Classifier (SVC), with hyperparameter tuning and 5-fold cross-validation for performance evaluation. Feature importance was analyzed using Shapley Additive Explanations (SHAP) and permutation importance to enhance interpretability. Performance metrics included accuracy, precision, recall, F1-score, and Area Under the Curve (AUC) scores. Although the models were developed using a large dataset specifically from keloid patients, it is contemplated that similar approaches with smaller data could yield useful ML models for predicting response to steroid therapy for keloid and for other conditions as well.

### METHODS

Dataset. A comprehensive questionnaire was designed to survey a large, comprehensive cohort of patients diagnosed with keloid disorder. Participants were invited to complete the questionnaire by visiting the study’s website, www.KeloidSurvey.com. After reviewing the consent form, adult participants provided electronic consent, while parents could consent on behalf of their minor children. The questionnaire collected detailed information, including patient demographics, family history, the extent and distribution of keloid lesions, prior treatments, and responses to those treatments. To ensure data integrity, access to the survey was restricted to one entry per computer IP address. Participants had the option to skip questions that were either not applicable or that they preferred not to answer. An analysis of participants perceptions regarding the effectiveness of steroid injections has been published [1’]. The dataset used for this study was accessed in May 2024.

Feature Description. In FIG. 8, subpanel A to subpanel I, a description of the dataset and descriptive analyses are provided covering participant demographics, age of keloid onset, ethnicity, gender distribution, Race of patients, triggering factors, lesion distribution patterns, and lesion appearance and shape.

Gender. The data in FIG. 8, subpanel A shows that nearly twice as many female patients as male patients were diagnosed with keloids, which aligns with previous research suggesting that female sex may be a potential risk factor for keloid formation. Studies have indicated that keloid onset often occurs during puberty for both sexes, with females showing a higher prevalence, especially before age 15. This female predominance may be linked to physiological factors, including hormonal influences, and warrants further investigation into the role of sex hormones in keloidogenesis [2’].

Race and nationality. Participants were asked to indicate their ethnic background, and FIG. 8, subpanel B, represents the distribution of various ethnic groups among the respondents. Although the data may not fully represent the actual ethnic distribution of keloids in the broader population, the figure shows a higher incidence of keloids among African American participants. This observation is consistent with previously published studies, which have reported a greater predisposition to keloids in this group [4’,] [5’].

Age. Participants were requested to provide their current age, as well as the age at which they first noticed the development of a keloid. FIG. 8, subpanels D and E, respectively, illustrate both the participants’ ages at the time of completing the survey and the age at which their keloid initially appeared. As shown in this figure, most patients in our cohort showed keloid onset before age 20, with many cases starting as early as age 8. This is consistent with previous reports, suggesting that individuals predisposed to keloids tend to develop them early, particularly during adolescence [2’], [6’].

Intralesional Triamcinolone Acetonide Injections. Participants were asked for the total number of steroid injections they had received for their keloids, which is shown in FIG. 8, subpanel F. The survey used the term steroid injection instead of ILTA, as it was presumed that participants might not be familiar with the medical terminology [8’]. The survey also presumed that all patients had been treated with either TA or a similarly potent steroid. Additionally, participants were asked to recall how frequently they received these injections. The survey relied on patients’ own recollections and understanding of the treatments they underwent. However, the survey did not inquire about the specific type of steroid or the dosage administered. Notably, a significant number of participants reported receiving over 10 injections, often monthly. The effectiveness of ILTA was evaluated by asking participants to select only one response to each of three multiple-choice questions, which are quoted below exactly as they appeared in the survey, and the responses are recorded [18’].

Pattern of Distribution of Keloid Lesions: Participants were requested to provide detailed information regarding the distribution of their keloid lesions across their skin. The data shows FIG. 8, subpanel F, that the chest and shoulder areas are the most affected, while the ankle and forehead are the least. This distribution is consistent with other studies, suggesting that keloids are more likely to form in regions of high skin tension and frequent movement [7’].

Keloid triggering factors: Participants were asked to share information about the factors that potentially triggered the formation of their keloids. According to the data that is shown in FIG. 8, subpanel G, the participants, acne was the most frequently reported trigger, with skin injury and surgery being the next most common factors. These findings are consistent with previously published data [3’].

Appearance and Shape of Keloid Lesions. Participants were asked to describe the shape and appearance of their keloid lesions. To assist with this, an online reference guide was provided, offering categories such as flat, nodular, linear, massive, pedunculated, and superficially spreading FIG. 8, subpanel H. Representative examples of different keloid shapes are shown in FIG. 8, subpanel I.

Changes in keloid size over time. As part of the survey, participants were asked to compare the current size of their keloids to their size 1, 2, 5, and 10 years ago [18’]. Keloid size compared to 1, 2, 5, and 10 years ago: Participants were also asked to compare the size of their keloids 1-10 years after beginning steroid treatment, with the responses providing insights into how the size of their keloids changed over this period [18’].

Assessment of treatment response to steroid therapy: Participants were asked to evaluate the effectiveness of steroid therapy for their keloid lesions [18’]. Their responses include perceived outcomes (no improvement, worsening, improvement, or complete cure), changes in lesion size following treatment (i.e., final outcome of steroid injections), and classification of patients as either sensitive or resistant to steroid injections.

Feature Engineering. From the self-reported keloid patient dataset described above, the following variables were selected for analysis: gender, race, keloid shape, patient age, age at keloid onset, keloid history at 1, 2, 5, and 10 years prior, keloid location, keloid size changes over 1, 2, 5, and 10 years, trigger factors, and number of steroid injections administered.

After filtering the dataset to focus only on steroid therapy-related data, features with significantly fewer rows were removed from the analysis to ensure robustness. Subsequently, the remaining numerical features were standardized to a range of 0 to 1 to address the disparity in value ranges and optimize model performance.

Categorical features were then one-hot encoded. One-hot encoding converts categorical variables into a binary matrix representation, enabling ML algorithms to interpret the data more effectively. Each category is represented as a separate binary feature, ensuring that no ordinal relationships are assumed between categories. Following these steps, nine categories of features with different attributes remained FIG. 4A on which our ML models were built.

Handling Class Imbalance. Class imbalance is a common issue in many ML tasks, particularly in clinical datasets where the number of instances in one class significantly outnumbers those in another. This imbalance can lead to biased model performance, where the model becomes more proficient at predicting the majority class while underperforming on the minority class. To address this, various resampling techniques can be applied to balance the class distribution, improving the model’s ability to generalize to both classes. Synthetic Minority Over-sampling Technique (SMOTE) is a widely used method for addressing class imbalance. It generates synthetic examples of the minority class by interpolating between existing minority class instances. Specifically, SMOTE selects two or more similar instances in the minority class and creates a new synthetic instance along the line segment joining them. This approach helps to increase the representation of the minority class without simply duplicating the existing instances, thus providing a more diverse training set for the model. Edited Nearest Neighbors (ENN) is an under-sampling technique used to clean the dataset by removing noisy and borderline examples. ENN removes instances from the dataset that are misclassified by their k-nearest neighbors (k=3 used).

This may help in eliminating potential outliers and instances that might be ambiguously labeled, leading to a more distinct class boundary. SMOTEENN is a hybrid approach that combines the strengths of SMOTE and ENN. It first applies SMOTE to generate synthetic instances of the minority class and then applies ENN to remove noisy and borderline examples.

This combination helps to not only balance the class distribution but also enhance the quality of the training set by reducing noise and improving the class boundaries. For the dataset, the study evaluated several sampling strategies, and SMOTEENN delivered the best performance according to our evaluation metrics. This combination of generating synthetic instances and cleaning the dataset effectively handled the class imbalance in our keloid patient dataset, resulting in a more robust and generalizable model for predicting response to steroid therapy.

Data Splitting and Evaluation Metrics. To develop reliable ML models and evaluate their performance on unseen data, it is essential to split the dataset into training and testing subsets. This approach ensures that models are trained on one portion of the data (the training set) while their prediction accuracy is evaluated on a separate portion (the test set). For this purpose, the dataset was first randomly shuffled, and a 5-fold cross-validation technique was applied. This method involves dividing the dataset into five equal parts. During each iteration, one part is set aside as the test set, while the remaining four folds are used to train the model. This process is repeated five times, with each fold serving as the test set exactly once. Consequently, the benchmark results reported in the following sections are the averages of these five iterations. The predictive performance of the ML models was evaluated using several metrics. Accuracy, which measures the ratio of correctly predicted instances to the total instances, was used to provide a general measure of model performance. Precision, the ratio of true positive predictions to the total predicted positives, indicates the accuracy of positive predictions. Recall, the ratio of true positive predictions to the total actual positives, reflects the model’s ability to identify all relevant instances. The F1 score, which is the harmonic mean of precision and recall, offers a single metric that balances both concerns.

Additionally, the F0.5 score was utilized to evaluate performance, as it places greater emphasis on precision. This choice was made because false positives are particularly costly in our context. A false positive result would incorrectly indicate the need for a steroid injection, leading to unnecessary treatment for the patient. The ROC-AUC (Receiver Operating Characteristic Area Under the Curve) metric was employed to measure the model’s ability to distinguish between classes, with higher values indicating better performance. Lastly, the PR-AUC (Precision-Recall Area Under the Curve) metric was used to assess the trade-off between precision and recall across different threshold settings, which is particularly useful for imbalanced datasets. By using these metrics, the study ensure a comprehensive evaluation of the models, capturing their performance across various critical aspects of the task at hand, and understanding the strengths and weaknesses of each model, guiding further improvements and optimizations.

Machine Learning (ML) models. To predict the response to steroid therapy in keloid patients, the study applied a range of ML models (Table 1). Given the complexity and variability in keloid lesion formation, it was essential to explore different models to capture non-linear and intricate relationships within the data. Each model offers unique advantages in terms of classification capability and generalizability. By systematically comparing these models, the study aimed to identify the one best suited for this predictive task.

Random Forest (RF). RF, an ensemble learning algorithm, excels in regression applications [9’]. RF integrates bagging and bootstrap aggregating techniques. During bagging, RF generates multiple decision trees simultaneously, each trained on a randomly selected subset of the dataset, allowing for replacement. This strategy enhances tree diversity, mitigates overfitting, and improves generalization. Bootstrap aggregating involves sampling with re- placement from the original dataset to create multiple subsets for training. This approach ensures that each decision tree in the RF ensemble encounters different data instances, bolstering the model’s robustness and decreasing variance. During training, each tree in the RF ensemble independently predicts the target variable. For regression tasks, RF combines all tree predictions to form a final prediction, typically by averaging their outputs. This ensemble methodology often results in better predictive performance compared to single decision trees. A significant benefit of RF is its ability to handle high- dimensional datasets with numerous features, as it automatically selects a subset of features for each split, thus reducing overfitting risk and enhancing computational efficiency.

Gradient Boosting (GB) Trees. GB is another ensemble technique leveraging decision trees for predictions [10’]. Unlike RF, GB uses shallow decision trees, or weak learners, and combines their outputs during training. GB builds trees sequentially, with each new tree aiming to correct errors made by its predecessors. By iteratively refining these errors, GB constructs a strong predictive model over time. A key strength of GB is its ability to uncover complex relationships in the data, as the model continuously learns from its mistakes and improves predictions. Additionally, GB is less prone to overfitting compared to deep decision trees, making it suitable for various regression tasks. However, GB typically demands more computational resources and fine-tuning than Random Forest, given its sequential learning process. Despite this, GB often delivers superior predictive performance, particularly on structured datasets with intricate patterns [11’].

Neural Network (NN). NNs, a subset of ML, underpin deep learning techniques. Inspired by the human brain’s biological structure, NNs consist of interconnected neurons that communicate. These networks usually have an input layer, one or more hidden layers, and an output layer. During training, NNs undergo an iterative process where initial random weights are adjusted via backpropagation until the model minimizes the loss function, converging to a global minimum. By continuously fine-tuning weights based on observed errors, NNs can effectively learn complex data patterns and relationships. NNs are highly valued for their capacity to handle large and varied datasets and to learn hierarchical data representations. They are particularly effective in image and speech recognition, natural language processing, and other tasks. However, training deep neural networks can be computationally demanding and may require substantial labeled data. Nonetheless, NNs lead many state-of-the-art ML applications, driving advancements across numerous fields.

XGBoost. XGBoost, or Extreme Gradient Boosting, is an advanced variant of Gradient Boosting algorithms, incorporating regularization techniques like L1 and L2 for better generalization [12’]. Unlike traditional gradient-boosted trees, XGBoost offers superior speed, high performance, and the ability to conduct parallel tree boosting. Its efficiency makes XGBoost suitable for large-scale datasets and computationally intensive tasks. XGBoost is popular for its robustness and versatility across various ML applications. In the study, XGBoost is also used for feature importance analysis and SHAP (SHap- ley Additive exPlanations) analysis. These techniques help practitioners understand the relative importance of input features and the model’s decision-making process. This dual functionality enhances XGBoost’s interpretability, making it a valuable tool for both predictive modeling and interpretability in ML tasks.

Support Vector Classifier (SVC). Support Vector Machines (SVMs) are a class of ML algorithms used for both regression and classification tasks. For the classification task, SVMs predict by finding a hyperplane that separates data points of different categories by maximizing the distance, called the margin, between the nearest data points and the decision boundary [13’]. For the regression task, SVMs provide a decision boundary at a distance from the original hyperplane such that the data points closest to the hyperplane, called support vectors, are within that boundary line.

The instant study utilized the SVC, a SVM variant for classification. SVC finds the optimal hyperplane by focusing on support vectors, improving generalization to new data. Kernel functions allow SVC to handle non-linear relationships, making it versatile for various classification problems. SVC is effective in high-dimensional spaces and robust to overfitting, particularly when features outnumber samples.

Logistic Regression (LR). LR is a widely-used statistical method for binary classification tasks [10’]. LR models the probability of a binary outcome by fitting a logistic function to the input features. This approach transforms the linear combination of features into a probability score between 0 and 1. The model’s coefficients are estimated using maximum likelihood estimation, and the resulting probabilities are used to classify observations into one of two categories. One of the strengths of LR is its interpretability, as the coefficients can provide insights into the influence of individual features on the outcome. Despite its simplicity, LR can perform well on a variety of datasets, particularly when the relationship between the features and the outcome is approximately linear. However, LR may struggle with complex, non-linear relationships and typically requires feature scaling and careful handling of multicollinearity among predictors.

CatBoost. CatBoost is a gradient boosting algorithm developed by Yandex, designed specifically to handle categorical features effectively [14’]. Unlike many other algorithms that require pre-processing of categorical data into numerical form, CatBoost can directly process categorical variables, preserving their structure and relationships. This capability often leads to better performance and easier model deployment. CatBoost employs ordered boosting, a variant of gradient boosting that reduces overfit- ting and variance by processing data in a permutation-driven manner. This approach helps in maintaining the robustness of the model. CatBoost is known for its fast training times, ease of use, and strong performance on a wide range of datasets, making it a popular choice in the ML community.

Bagging. Bagging, short for Bootstrap Aggregating, is an ensemble learning technique that enhances the stability and accuracy of ML algorithms [15’]. Bagging involves creating multiple versions of a predictor by training each version on a different bootstrap sample from the original dataset. The final prediction is obtained by averaging (for regression) or voting (for classification) across all predictors. This method reduces variance and helps prevent overfitting, especially for high-variance models like decision trees. By training multiple models on different subsets of the data, Bagging produces a more robust and generalizable predictive model. One of the notable advantages of Bagging is its simplicity and ability to improve the performance of many base models without requiring significant modifications.

AdaBoost. AdaBoost, short for Adaptive Boosting, is an ensemble technique that combines the out- puts of weak learners to create a strong classifier [16']. Unlike Bagging, which trains multiple models independently, AdaBoost builds models sequentially. Each new model focuses on the instances that the previous models misclassified, adjusting their weights to improve performance on these hard-to-classify examples.

AdaBoost works by assigning higher weights to incorrectly classified instances and lower weights to correctly classified ones. This iterative process continues until a predetermined number of models are trained or no further improvement is observed. AdaBoost is known for its simplicity, effectiveness, and ability to reduce both bias and variance. However, it can be sensitive to noisy data and outliers, which may affect its performance if not properly managed. For the keloid patient dataset, the study implemented each of these models and evaluated their performance using comprehensive metrics. This thorough approach allowed us to identify the most effective model for predicting response to steroid therapy, after accounting for the specific challenges posed by the data.

Hyperparameter Optimization. Hyperparameter optimization (Supplementary Table S1) is crucial in ML to finetune models for optimal performance. For Random Forest, the hyperparameters were tuned including n estimators, max depth, min samples split, min samples leaf, max features, and bootstrap. These parameters control the number of trees in the forest, the maximum depth of each tree, the minimum number of samples required to split an internal node, the minimum number of samples required to be at a leaf node, the number of features to consider when looking for the best split, and whether bootstrap samples are used when building trees.

SVC optimization focused on parameters such as C, gamma, and kernel. The C parameter balances the trade-off between achieving a low training error and minimizing model complexity, gamma defines the influence of a single training example and affects the flexibility of the decision boundary, and kernel specifies the kernel type used in the algorithm (linear, polynomial, sigmoid, or radial basis function), impacting the decision function shape and model flexibility.

Logistic Regression’s hyperparameters, including C, solver, penalty, and max-iter, were optimized to enhance model regularization, optimization method, regularization type, and convergence criteria, respectively. These parameters collectively govern model fitting and performance optimization, crucial for achieving robust logistic regression models.

Gradient Boosting tuned parameters such as n estimators, learning rate, max depth, subsample, and min samples split. These parameters control the number of boosting stages, the shrinkage of the contribution of each tree, the maximum depth of each tree, the fraction of samples used for fitting each tree, and the minimum number of samples required to split an internal node, collectively enhancing model fitting and generalization through boosting.

Neural Network optimization involved parameters like hidden layer sizes, activation, solver, alpha, learning rate, and max-iter. These parameters define the network architecture, activation functions for hidden layers, weight optimization strategy, regularization strength, learning rate schedule, and maximum number of iterations, influencing the neural network’s learning dynamics and performance.

XGBoost parameters, including n estimators, learning rate, max depth, subsample, and colsample bytree, were tuned to optimize the number of boosting rounds, control overfitting by learning rate adjustment, limit tree depth and instance subsampling, and select the fraction of features used per tree, enhancing prediction accuracy and efficiency through boosting.

CatBoost optimized iterations, learning rate, depth, and l2 leaf reg parameters. These parameters control the number of boosting iterations, step size during optimization, tree depth, and leaf regularization, respectively, optimizing boosting process characteristics to improve model training and performance.

Bagging’s hyperparameters, including base estimator max depth and n estimators, were tuned to restrict base model depth and determine the number of base estimators, influencing ensemble complexity and robustness for improved prediction accuracy.

AdaBoost’s parameters, n estimators and learning rate, were optimized to define the maximum number of boosting stages and weight the contribution of each classifier, respectively, governing boosting round execution and impact on model learning and prediction.

The optimization details, along with the best-performing hyperparameter values, are summarized in Supplementary Table S1. This table offers a comprehensive overview of the key hyperparameters for each model and their optimal values.

Feature Importance Analysis. In the study, two complementary methods, SHAP (Shapley Additive Explanations) and Permutation Importance, were used to evaluate feature importance and interpret the model’s predictions. These methods offer distinct but valuable perspectives on the relative influence of input features.

SHAP (Shapley Additive Explanations). SHAP, grounded in cooperative game theory, was employed for comprehensive feature importance analysis [17’]. SHAP assigns an importance value to each feature by calculating its contribution to the model’s predictions, based on all possible combinations of features. This makes it particularly useful for capturing interactions between features, which is crucial in models where features collectively influence predictions. The framework ensures that the sum of the feature importance values equals the model’s total output, providing both mathematically rigorous and interpretable results.

SHAP allows for global interpretability, identifying the most influential features across the entire dataset, and local interpretability, examining the specific impact of features on individual predictions. This dual capability is essential in understanding how different combinations of clinical features contribute to model predictions.

In the study, SHAP revealed the importance of key clinical features such as gender, age, and growth history of keloid lesions, not only as individual predictors, but also in their interactions with other features. This deeper understanding of the model’s internal mechanics enabled us to refine the model, focusing on the most critical features and ensuring that the predictions were consistent with clinical knowledge. Using SHAP, the study was able to maintain a transparent and interpretable model, aligning predictive insights with clinical expertise and strengthening the credibility of the findings.

Permutation Importance. To complement SHAP, the study employed permutation importance to provide a more global measure of feature importance. Permutation importance evaluates how much each feature contributes to the overall model performance by randomly shuffling its values and observing the resulting drop in accuracy [9’]. This method provides a straightforward assessment of how the model depends on each feature, independent of interactions.

The strength of permutation importance lies in its simplicity, offering a clear, global view of feature influence. Unlike SHAP, which excels at capturing feature interactions, permutation importance focuses on each feature’s individual impact. Together, these methods offer a comprehensive perspective on feature importance, balancing the nuanced interactions identified by SHAP with the direct, global influence revealed by permutation importance.

Permutation importance was calculated by measuring the model’s accuracy before and after shuffling each feature. A significant drop in accuracy indicated the feature’s importance in making predictions. Like SHAP, permutation importance was also aggregated at the group level to evaluate clinically relevant feature sets. By combining SHAP and permutation importance, the study gained a more holistic understanding of feature importance.

SHAP provides the nuance of feature interactions and individualized predictions, while permutation importance offers a straightforward and intuitive ranking of feature influence on global model performance. Together, these methods ensure that the study not only understand which features the model relies on but also how these features interact to drive clinical predictions.

### Results

In this study, the predictive capabilities of the ML models applied to the dataset are presented. A variety of models were trained and evaluated using a range of performance metrics, including accuracy, precision, recall, F1 score, Receiver Operating Characteristic Area Under the Curve (ROC-AUC), F0.5 score, and Precision-Recall Area Under the Curve (PR-AUC). Additionally, cross-validation techniques were employed to ensure reliable and robust results. Several visualization techniques, including heatmaps and spider plots, were used to offer a comparative view of model performance across all metrics.

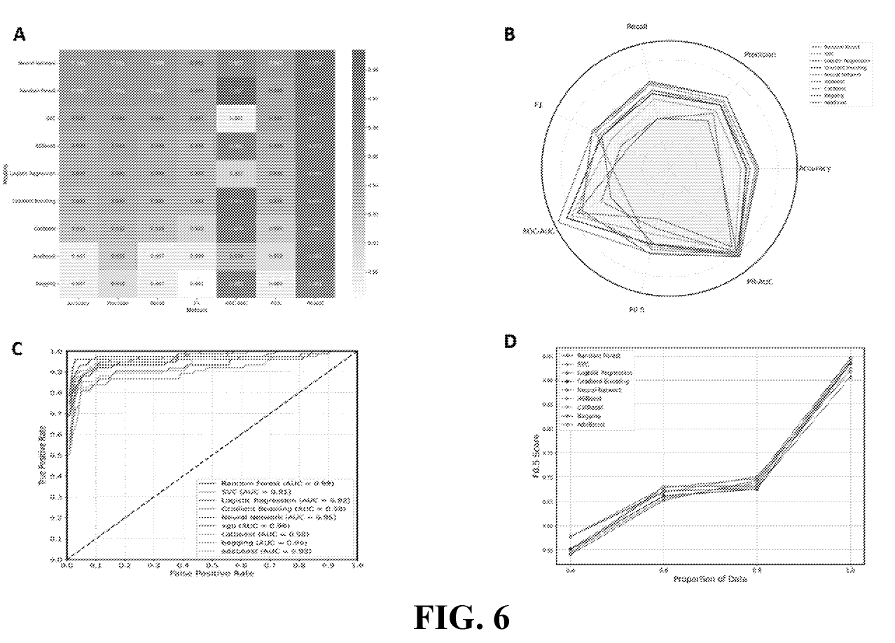

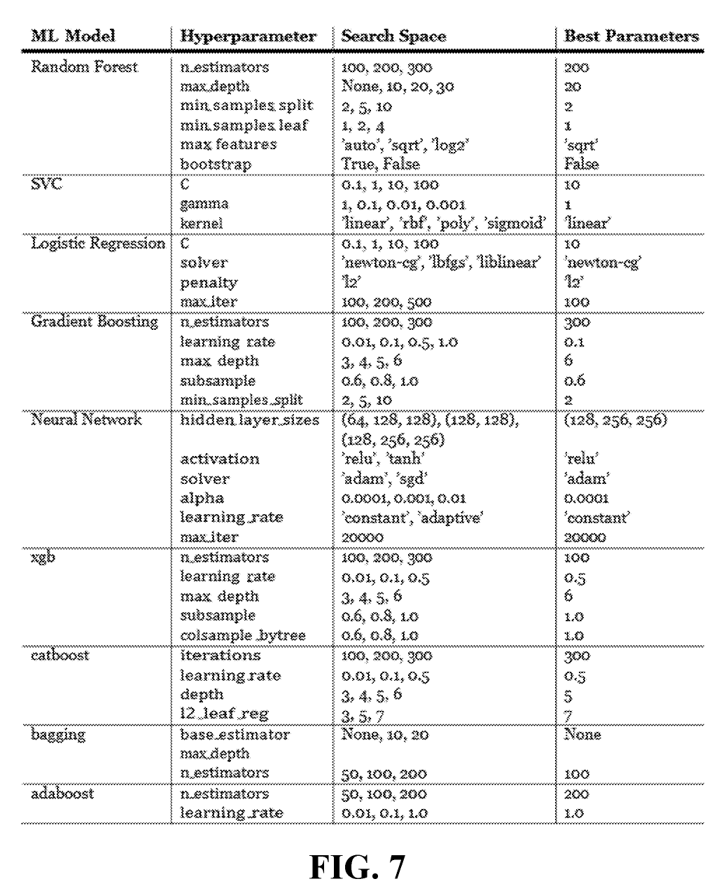

Performance of different ML models in predicting response to steroid therapy for keloids. FIG. 6 shows a summary of the performance of various ML models, including accuracy, precision, recall, F1 score, ROC-AUC-OVR, F0.5 score, and PR-AUC. FIG. 6, subpanel A and subpanel B, visually compare model performances via a heatmap and a spider plot. NN achieved the highest accuracy (0.949 ± 0.013), precision (0.951 ±0.012), recall (0.949 ± 0.013), and F1 score (0.945 ± 0.015). Its ROC-AUC of 0.957 ± 0.041 further confirms strong class distinction. RF closely followed, achieving 0.947 ± 0.025 accuracy and a higher ROC-AUC of 0.987 ± 0.024. Support Vector Classifier (SVC) showed good accuracy (0.945 ± 0.016) but lower ROC-AUC (0.907 ± 0.052). Gradient Boosting (GB) and CatBoost performed well, with accuracies of 0.935 ± 0.024 and 0.929 ± 0.023 and ROC-AUC values of 0.976 ± 0.041 and 0.970 ± 0.030, respectively. XGBoost (accuracy 0.939 ± 0.030, ROC-AUC 0.962 ± 0.054) and Bagging (accuracy 0.907 ± 0.043) also showed solid performance. PR-AUC values remained consistently high across models, mostly above 0.95, confirming strong precision-recall balance. While NN showed slightly better precision, recall, and F1 scores, RF demonstrated comparable or superior ROC-AUC, indicating robust class-separation. In practice, NN may be preferred for scalability, while RF for easier interpretability and feature analysis.

ROC-AUC comparison of different ML models. The performance of the ML models was evaluated using ROC curves, which are FIG. 6, subpanel C. Each model is represented by a distinct line on the plot, illustrating the relationship between the true positive rate (sensitivity) and the false positive rate (1−specificity) across different classification thresholds. A model that performs better will have a curve that is closer to the top-left corner, indicating higher sensitivity with fewer false positives. The AUC was calculated for each model to quantify performance, with higher AUC values signifying more effective models.

The AUC values for the models are as follows: RF (0.99), SVC (0.91), Logistic Regression (0.92), GB (0.98), NN (0.95), XGBoost (0.96), Cat-Boost (0.98), Bagging (0.96), and AdaBoost (0.93). Among these, RF showed the highest AUC scores, indicating excellent classification performance with near-perfect sensitivity and specificity. All models exhibit strong performance, with AUC values above 0.90, reflecting their effectiveness in accurately classifying the dataset.

Effect of training set size on model accuracy. The impact of training set size on regression accuracy was assessed by randomly selecting 40%, 60%, 80%, and 100% of the training data, followed by 5-fold cross-validation across ML models. FIG. 6, subpanel D, shows that F0.5 scores improve with larger training sets, indicating higher prediction accuracy. Model interpretability and importance of individual features.

Feature importance was analyzed using SHAP and permutation methods to identify key clinical and demographic factors influencing model predictions and steroid therapy outcomes in keloid patients.

SHAP Results. The global SHAP bar plot (FIG. 5, subpanel A) shows the mean absolute SHAP values for each feature group, highlighting patient gender and keloid morphology as the strongest predictors of steroid therapy outcomes. Female patients were more likely to be predicted as sensitive to treatment, while males tended toward resistance. Similarly, lesion morphology—flat, nodular, or massive—strongly influenced predictions, with flat and nodular lesions associated with sensitivity and massive lesions with resistance.

Growth history also had a major impact. Patients whose keloids continued growing over 1, 2, 5, or 10 years were more likely to be predicted as resistant, while other factors like number of steroid injections, age of onset, and lesion location contributed moderately. Initial models included growth history, intuitively a strong predictor since persistent growth after multiple steroid injections often signals resistance. However, because clinical scenarios often involve treatment-naïve patients or missing records, feature ablation analysis was performed. Removing growth history, number of injections, or both led to a modest drop in model accuracy (from ~95% to ~90%), demonstrating that models remained clinically valuable without these features. The SHAP summary plot (FIG. 5, subpanel (B)) provided patient-level insights, with each dot representing how a feature shifted predictions toward sensitivity (positive SHAP value) or resistance (negative SHAP value). Red dots indicated higher feature values (e.g., female gender), blue lower values (e.g., male gender). Analysis showed that flat or nodular morphology and female gender favored sensitivity, while massive morphology and growth history favored resistance. Finally, SHAP interaction analysis (FIG. 5, subpanel (C)) explored joint effects of age and gender. Younger female patients showed strong positive interaction effects (greater likelihood of steroid sensitivity), but this reversed with aging, where older females trended toward resistance. In contrast, younger males were more resistant, but older males showed interaction values moving toward zero or slightly positive, suggesting reduced resistance with age.

These interactions highlight that feature relationships evolve with patient demographics, underscoring the importance of analyzing feature combinations rather than isolated effects.

Permutation Importance. To complement SHAP analysis, the study applied permutation importance to assess each feature’s standalone contribution by measuring any reduction in model performance when feature values were shuffled. The group-level permutation plot (FIG. 5, subpanel (D)) confirmed that patient gender and keloid morphology are the most important factors individually. While age showed a strong influence in SHAP analysis through interaction with gender, permutation results revealed low standalone importance, suggesting age impacts predictions mainly through interactions. Together, these analyses provide a comprehensive understanding of how clinical and demographic factors influence model predictions, aiding in personalizing steroid therapy for keloids.

Web-based tool for clinicians to predict steroid response. The study developed a web-based tool for clinicians to predict steroid response for keloids at the point of care. Clinicians can input the relevant data to obtain a prediction regarding the use of steroids for keloid therapy in individual patients.

Discussion. The study evaluated nine ML models to predict keloid patient responses to corticosteroid therapy, assessing performance across seven metrics. NN and RF achieved top-tier results, with NN showing slightly higher accuracy and F1 score, while RF demonstrated marginally higher ROC-AUC. Both models offer strong predictive power; however, NN was selected as the primary model for its consistent performance and scalability. Clinical and demographic data analysis identified gender as the strongest predictor, with female patients more likely to respond favorably to therapy. Notably, women under 45 showed higher sensitivity to ILTA therapy, whereas older women exhibited resistance, potentially reflecting hormonal changes post-menopause. These findings highlight the importance of gender-specific factors in predicting steroid treatment outcomes for keloid disorder.

The findings are consistent with published studies suggesting a gender bias in response to steroid therapy. Several studies have reported that females respond less favorably to corticosteroid treatments compared to males -. For example, older female patients with inflammatory bowel disease (IBD) are more likely to exhibit prednisone resistance or dependence compared to younger females and males . This gender disparity in steroid response may be underpinned by the sexually dimorphic effects of glucocorticoids. Crosstalk between glucocorticoid and sex steroid hormones significantly impacts key processes like metabolism and immune regulation . For instance, glucocorticoid receptor (GR) signaling can be modulated by estrogen receptor (ERα and ERβ) pathways, suggesting a biological basis for the observed differences in therapeutic outcomes between males and females . Furthermore, in systemic lupus erythematosus (SLE), a disease with a striking female predominance (9:1 female-to-male ratio), estradiol-mediated activation of ERα has been implicated in disease progression. Blocking Erα action with antagonists such as Fulvestrant has been shown to improve disease indicators, pointing to the significance of steroid receptor crosstalk in disease mechanisms . These insights into the interplay between GR and ERα pathways underscore the need to consider gender as a critical factor in optimizing steroid-based therapies. Addressing these differences is essential for improving outcomes and tailoring treatments for conditions with significant gender disparities in steroid response.

The morphology of keloid lesions emerged as the second most important feature in predicting steroid sensitivity, with flat and nodular keloid lesions showing greater responsiveness compared to massive keloid lesions. While a previous study focused on keloid lesion contour as a predictor of steroid response, finding that patients with higher contour scores were more likely to respond to corticosteroid injections within a 3-month follow-up period , the findings may not necessarily contradict this. Contour and morphology are related parameters, with higher contour (in the Z-plane) potentially correlating with smaller keloid lesions in the X-Y plane, leading to a more nodular appearance. The instant study, however, utilized self-reported patient data collected over a longer timeframe, capturing the long-term impact of keloid behavior and response to steroid treatment. This broader perspective may provide a more comprehensive understanding of the relationship between keloid morphology and steroid sensitivity.

The growth history of keloids was the third most important feature in predicting response to steroids, indicating that patients with ongoing keloid growth for 1 to 10 years were more likely to exhibit steroid resistance. This finding is consistent with data from online keloid disorder management resources, which similarly highlight that persistent growth over extended periods is associated with poor response to steroid therapy . Removing the growth history of keloids from the models resulted in little or no decrease in the predictive values of the models.

Age is also one of the important factors in steroid sensitivity in keloid patients. This finding aligns with existing evidence suggesting that age plays a significant role in corticosteroid response across various medical conditions. In general, younger patients often exhibit more robust responses to steroids compared to older individuals, which correlates with the duration of the illness, and age of the keloids. This phenomenon may be attributed to age-related changes in immune function, metabolism, and tissue repair mechanisms, which can influence how steroids exert their anti-inflammatory and immunosuppressive effects. For asthma and autoimmune diseases, studies have reported decreased steroid efficacy in older patients [8], -, potentially due to age-related reductions in glucocorticoid receptor sensitivity or altered inflammatory pathways. The observed relationship between age and steroid sensitivity in keloid patients may similarly reflect underlying biological changes that affect fibroblast proliferation, collagen synthesis, and overall wound healing processes. Currently, there are no clinical data on the relationship between age and steroid resistance in keloid patients. Future clinical studies should address this gap, as understanding age-related differences is crucial for clinicians to make informed decisions about selecting the most appropriate therapies.

The age of onset and location of keloids, as well as triggering factors were also significant predictors of steroid sensitivity. Earlier onset of keloid lesions correlated with varying levels of treatment response, suggesting that the age at which keloid lesions first develop may influence their sensitivity to steroid therapy. The initial trigger for the formation of keloid lesions, such as acne, surgery, or injury, highlighted by SHAP analysis, also played a role in predicting outcomes, with different triggers potentially affecting keloid behavior and response to treatment. Furthermore, the anatomical location of the keloid influenced responsiveness, as certain locations were more likely to show favorable outcomes on steroid therapy compared to others. These findings emphasize the multifactorial nature of keloid disorder and the importance of considering these clinical variables when developing personalized therapies.

Indeed, the study demonstrated that ML models can improve the prediction of response to intralesional steroids in keloid patients and also through a web-based interface. By identifying key predictors such as gender, keloid lesion morphology, growth history, and other clinical factors, the ML model can provide insights that can guide more effective and personalized treatment strategies. The findings underscore the importance of considering patient-specific characteristics in clinical practice and highlight the need for further research to validate these results and explore the underlying biological mechanisms. Such advancements will help optimize therapeutic decisions and improve outcomes for keloid patients. Although the current models were developed using a large dataset specifically from keloid patients, it is contemplated that similar approaches with less data would yield useful ML models for predicting response to steroid therapy for other conditions as well.

### Discussion

Keloid disorders are non-lethal fibroproliferative skin tumors/lesions characterized by excessive fibroblast proliferation and overproduction of extracellular matrix, particularly type I collagen [1]. Lesions extend beyond original wound boundaries and often cause pain, itching, and significant cosmetic and psychosocial distress. Management remains challenging due to high recurrence rates [2]. Intralesional triamcinolone acetonide (ILTA) is a mainline therapy for keloids, and results in dose-dependent reductions in inflammation, fibroblast activity, and collagen synthesis [3], [4]. Since its first reported efficacy in 1960 [4] ILTA remains widely used, with ongoing studies supporting its role in keloid regression [3].

Despite the widespread use of ILTA for keloids, patient response is inconsistent, with only about two-thirds of the patients benefiting to different extents, while a significant fraction experiences a worsening of symptoms [5]-[7]. Steroid resistance is observed in other conditions, including pediatric nephrotic syndrome [8], asthma , and inflammatory bowel diseases [9], highlighting variability in treatment response and the need for predictive tools.

Machine learning (ML) offers promising solutions in personalized medicine, enabling prediction of treatment outcomes by analyzing complex clinical datasets. ML has been successfully applied in diagnostics and treatment outcome prediction, enabling more tailored interventions [10]. By analyzing patient data, including demographic, genetic, and clinical information, ML models can identify patterns that may not be apparent through traditional analysis [11]. In oncology, algorithms like random forests (RF) and neural networks (NN) predict survival outcomes [12], while in surgery, ML models have optimized treatment planning and improved patient safety [13].

The instant study applied ML tools to predict steroid treatment outcomes based on self-reported keloid patient survey data. By identifying patients unlikely to benefit from ILTA, clinicians can recommend alternative therapies, improving outcomes and reducing ineffective treatments.

### Example Artificial Intelligence (AI) and Machine Learning (ML) Models

Machine Learning. In addition to the machine learning features described above, the exemplary system can be implemented using one or more artificial intelligence and machine learning operations. The term “artificial intelligence” can include any technique that enables one or more computing devices or computing systems (i.e., a machine) to mimic human intelligence. Artificial intelligence (AI) includes but is not limited to knowledge bases, machine learning, representation learning, and deep learning. The term “machine learning” is defined herein to be a subset of AI that enables a machine to acquire knowledge by extracting patterns from raw data. Machine learning techniques include, but are not limited to, logistic regression, support vector machines (SVMs), decision trees, Naïve Bayes classifiers, and artificial neural networks. The term “representation learning” is defined herein to be a subset of machine learning that enables a machine to automatically discover representations needed for feature detection, prediction, or classification from raw data. Representation learning techniques include, but are not limited to, autoencoders and embeddings. The term “deep learning” is defined herein to be a subset of machine learning that enables a machine to automatically discover representations needed for feature detection, prediction, classification, etc., using layers of processing. Deep learning techniques include, but are not limited to, artificial neural networks or multilayer perceptron (MLP).

An artificial neural network (ANN) is a computing system including a plurality of interconnected neurons (e.g., also referred to as “nodes”). This disclosure contemplates that the nodes can be implemented using a computing device (e.g., a processing unit and memory as described herein). The nodes can be arranged in a plurality of layers, such as an input layer, an output layer, and optionally one or more hidden layers with different activation functions. An ANN having hidden layers can be referred to as a deep neural network or multilayer perceptron (MLP). Each node is connected to one or more other nodes in the ANN. For example, each layer is made of a plurality of nodes, where each node is connected to all nodes in the previous layer. The nodes in a given layer are not interconnected with one another, i.e., the nodes in a given layer function independently of one another. As used herein, nodes in the input layer receive data from outside of the ANN, nodes in the hidden layer(s) modify the data between the input and output layers, and nodes in the output layer provide the results. Each node is configured to receive an input, implement an activation function (e.g., binary step, linear, sigmoid, tanh, or rectified linear unit (ReLU) function), and provide an output in accordance with the activation function. Additionally, each node is associated with a respective weight. ANNs are trained with a dataset to maximize or minimize an objective function. In some implementations, the objective function is a cost function, which is a measure of the ANN’s performance (e.g., error such as L1 or L2 loss) during training, and the training algorithm tunes the node weights and/or bias to minimize the cost function. This disclosure contemplates that any algorithm that finds the maximum or minimum of the objective function can be used for training the ANN. Training algorithms for ANNs include, but are not limited to, backpropagation. It should be understood that an artificial neural network is provided only as an example machine learning model. This disclosure contemplates that the machine learning model can be any supervised learning model, semi-supervised learning model, or unsupervised learning model. Optionally, the machine learning model is a deep learning model. Machine learning models are known in the art and are therefore not described in further detail herein.

A convolutional neural network (CNN) is a type of deep neural network that has been applied, for example, to image analysis applications. Unlike traditional neural networks, each layer in a CNN has a plurality of nodes arranged in three dimensions (width, height, depth). CNNs can include different types of layers, e.g., convolutional, pooling, and fully-connected (also referred to herein as “dense”) layers. A convolutional layer includes a set of filters and performs the bulk of the computations. A pooling layer is optionally inserted between convolutional layers to reduce the computational power and/or control overfitting (e.g., by downsampling). A fully-connected layer includes neurons, where each neuron is connected to all of the neurons in the previous layer. The layers are stacked similarly to traditional neural networks. GCNNs are CNNs that have been adapted to work on structured datasets such as graphs.

Other Supervised Learning Models. A logistic regression (LR) classifier is a supervised classification model that uses the logistic function to predict the probability of a target, which can be used for classification. LR classifiers are trained with a data set (also referred to herein as a “dataset”) to maximize or minimize an objective function, for example, a measure of the LR classifier’s performance (e.g., an error such as L1 or L2 loss), during training. This disclosure contemplates that any algorithm that finds the minimum of the cost function can be used. LR classifiers are known in the art and are therefore not described in further detail herein.

A Naïve Bayes’ (NB) classifier is a supervised classification model that is based on Bayes’ Theorem, which assumes independence among features (i.e., the presence of one feature in a class is unrelated to the presence of any other features). NB classifiers are trained with a data set by computing the conditional probability distribution of each feature given a label and applying Bayes’ Theorem to compute the conditional probability distribution of a label given an observation. NB classifiers are known in the art and are therefore not described in further detail herein.

A k-NN classifier is an unsupervised classification model that classifies new data points based on similarity measures (e.g., distance functions). The k-NN classifiers are trained with a data set (also referred to herein as a “dataset”) to maximize or minimize a measure of the k-NN classifier’s performance during training. This disclosure contemplates any algorithm that finds the maximum or minimum. The k-NN classifiers are known in the art and are therefore not described in further detail herein.

A majority voting ensemble is a meta-classifier that combines a plurality of machine learning classifiers for classification via majority voting. In other words, the majority voting ensemble’s final prediction (e.g., class label) is the one predicted most frequently by the member classification models. The majority voting ensembles are known in the art and are therefore not described in further detail herein.

### Conclusion

Computer-executable instructions, such as program modules, being executed by a computer, may be used. Generally, program modules include routines, programs, objects, components, data structures, etc., that perform particular tasks or implement particular abstract data types. In its most basic configuration, the controller includes at least one processing unit and memory. Depending on the exact configuration and type of computing device, memory may be volatile (such as random-access memory (RAM)), non-volatile (such as read-only memory (ROM), flash memory, etc.), or some combination of the two. The controller may have additional features/functionality.

It should be understood that the various techniques described herein may be implemented in connection with hardware components or software components or, where appropriate, with a combination of both. Illustrative types of hardware components that can be used include Field-programmable Gate Arrays (FPGAs), Application-specific Integrated Circuits (ASICs), Application-specific Standard Products (ASSPs), System-on-a-chip systems (SOCs), Complex Programmable Logic Devices (CPLDs), etc. The methods and apparatus of the presently disclosed subject matter, or certain aspects or portions thereof, may take the form of program code (i.e., instructions) embodied in tangible media, such as floppy diskettes, CD-ROMs, hard drives, or any other machine-readable storage medium where, when the program code is loaded into and executed by a machine, such as a computer, the machine becomes an apparatus for practicing the presently disclosed subject matter.

Although exemplary implementations may refer to utilizing aspects of the presently disclosed subject matter in the context of one or more stand-alone computer systems, the subject matter is not so limited but rather may be implemented in connection with any computing environment, such as a network or distributed computing environment. Still further, aspects of the presently disclosed subject matter may be implemented in or across a plurality of processing chips or devices, and storage may similarly be implemented across a plurality of devices. Such devices might include personal computers, network servers, handheld devices, and wearable devices, for example.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an,” and “the” include plural referents unless the context clearly dictates otherwise. Ranges may be expressed herein as from “about” or “ 5 approximately” one particular value and/or to “about” or “approximately” another particular value. When such a range is expressed, other exemplary embodiments include from the one particular value and/or to the other particular value.

By “comprising” or “containing” or “including” is meant that at least the name compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, 4.24, and 5).

Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The following patents, applications, and publications, as listed below and throughout this document, are hereby incorporated by reference in their entirety herein.
[1] 1. Zhang, X., X. Wu, and D. Li, The Communication from Immune Cells to the Fibroblasts in Keloids: Implications for Immunotherapy. International Journal of Molecular Sciences, 2023. 24(20): p. 15475.
[2] Chike-Obi, C.J., P.D. Cole, and A.E. Brissett, Keloids: pathogenesis, clinical features, and management. 2009. p. 178--184.
[3] Atiyeh, B.S., Nonsurgical management of hypertrophic scars: evidence-based therapies, standard practices, and emerging methods. Aesthetic plastic surgery, 2007. 31: p. 468--492.
[4] Sexton, G.B., Local Injection of Triamcinolone Acetonide in the Management of Certain Skin Conditions:(Preliminary Report). Canadian Medical Association Journal, 1960. 83(26): p. 1379.
[5] Tirgan, M. Worsening of keloids after intralesional injections. in Journal of the American Academy of Dermatology. 2013. MOSBY-ELSEVIER 360 PARK AVENUE SOUTH, NEW YORK, NY 10010-1710 USA.
[6] Tirgan, M., Intralesional triamcinolone acetonide in the treatment of keloid lesions-can the treatment be harmful to some patients' results of an online survey. Int J Keloid Res, 2017. 1: p. 21-28.
[7] Muneuchi, G., et al., Long-term outcome of intralesional injection of triamcinolone acetonide for the treatment of keloid scars in Asian patients. Scandinavian journal of plastic and reconstructive surgery and hand surgery, 2006. 40(2): p. 111--116.
[8] Adcock, I.M., et al., Steroid resistance in asthma: mechanisms and treatment options. Current allergy and asthma reports, 2008. 8(2): p. 171-178.
[9] Coates, E., et al., Patient preferences and current practice for adults with steroid-resistant ulcerative colitis: POPSTER mixed-methods study. Health Technology Assessment (Winchester, England), 2022. 26(41): p. 1.
[10] Sidey-Gibbons, J.A.M. and C.J. Sidey-Gibbons, Machine learning in medicine: a practical introduction. BMC medical research methodology, 2019. 19: p. 1--18.
[11] Pfob, A., S.-C. Lu, and C. Sidey-Gibbons, Machine learning in medicine: a practical introduction to techniques for data pre-processing, hyperparameter tuning, and model comparison. BMC medical research methodology, 2022. 22(1): p. 282.
[12] Huang, Y., et al., Application of machine learning in predicting survival outcomes involving realworld data: a scoping review. BMC medical research methodology, 2023. 23(1): p. 268.
[13] Arjmandnia, F. and E. Alimohammadi, The value of machine learning technology and artificial intelligence to enhance patient safety in spine surgery: a review. Patient Safety in Surgery, 2024. 18(1): p. 11.
[14] Lucafò, M., et al., Gender may influence the immunosuppressive actions of prednisone in young patients with inflammatory bowel disease. Frontiers in Immunology, 2021. 12: p. 673068.
[15] Cvoro, A., et al., Cross talk between glucocorticoid and estrogen receptors occurs at a subset of proinflammatory genes. The Journal of Immunology, 2011. 186(7): p. 4354-4360.
[16] Rider, V., et al., Gender bias in human systemic lupus erythematosus: a problem of steroid receptor action? Frontiers in immunology, 2018. 9: p. 611.
[17] Lee, I., et al., Gender differences in prednisone adverse effects: Survey result from the MG registry. Neurology: Neuroimmunology & Neuroinflammation, 2018. 5(6): p. e507.
[18] Ud-Din, S., et al., Identification of steroid sensitive responders versus non-responders in the treatment of keloid disease. Archives of dermatological research, 2013. 305: p. 423-432.
[19] Juckett, G. and H. Hartman-Adams, Management of keloids and hypertrophic scars. American family physician, 2009. 80(3): p. 253-260.
[20] Jaiswal, A.K., et al., Short palate, lung, and nasal epithelial clone 1 (SPLUNC1) level determines steroid-resistant airway inflammation in aging. American Journal of Physiology-Lung Cellular and Molecular Physiology, 2022. 322(1): p. L102-L115.
[21] Borkar, N.A., C.K. Combs, and V. Sathish, Sex steroids effects on asthma: a network perspective of immune and airway cells. Cells, 2022. 11(14): p. 2238.

Reference List #2
[1’] Tirgan, M. Worsening of keloids after intralesional injections. in Journal of the American Academy of Dermatology, 2013. 68: p. AB68.
[2’] Chikage, N., Y. Hayasaka, and R. Ogawa, Sex Differences in Keloidogenesis: An Analysis of 1659 Keloid Patients in Japan. Dermatology and Therapy, 2019. 9: p. 747 - 754.
[3’] Wen-sheng, L., et al., Clinical and epidemiological analysis of keloids in Chinese patients. Archives of Dermatological Research, 2015. 307: p. 109-114
[4’] Swenson, A., et al., Natural History of Keloids: A Sociodemographic Analysis Using Structured and Unstructured Data. Dermatology and Therapy, 2024. 14(1): p. 131-149.
[5’] Tot, L., et al., Textbook on scar management: state of the art management and emerging technologies. 2020.
[6’] Ogawa, R., et al., The relationship between skin stretching/contraction and pathologic scarring: the important role of mechanical forces in keloid generation. Wound Repair and Regeneration, 2012. 20(2): p. 149--157.
[7’] McGinty, S. and W.J. Siddiqui, Keloid. 2018.
[8’] Tirgan, M., Intralesional triamcinolone acetonide in the treatment of keloid lesions-can the treatment be harmful to some patients? results of an online survey. Int J Keloid Res, 2017. 1: p. 21-28.
[9’] Breiman, L.
[10’] Friedman, J., T. Hastie, and R. Tibshirani, Additive logistic regression: a statistical view of boosting (with discussion and a rejoinder by the authors). The annals of statistics, 2000. 28(2): p. 337--407.
[11’] Hinton, G.E., S. Osindero, and Y.-W. Teh, A fast learning algorithm for deep belief nets. Neural computation, 2006. 18(7): p. 1527-1554.
[12’] Chen, T. and G. Carlos, “XGBoost: A Scalable Tree Boosting System.”. Proceedings of the 22nd ACM SIGKDD International Conference on Knowledge Discovery and Data Mining, 2016: p. 785–94.
[13’] Cortes, C. and V. Vapnik.
[14’] Prokhorenkova, L., et al., CatBoost: unbiased boosting with categorical features. Advances in neural information processing systems, 2018. 31.
[15’] Breiman, L., Bagging predictors. Machine learning, 1996. 24: p. 123--140.
[16’] Freund, Y. and R.E. Schapire, A decision-theoretic generalization of on-line learning and an application to boosting. Journal of computer and system sciences, 1997. 55(1): p. 119--139.
[17’] Lundberg, S.M. and S.-I. Lee, A unified approach to interpreting model predictions. Advances in neural information processing systems, 2017. 30
[18’] Zamani, Nina, et al. "Leveraging Machine Learning and Clinical Data to Predict Response to Intralesional Corticosteroids in Keloid Patients." medRxiv (2025): 2025-05.

What is claimed:

1. A system comprising:
a processor; and
a memory having instructions stored thereon, wherein execution of the instructions by the processor, causes the processor to:
receive input comprising three or more of:
gender for a patient,
shape of a keloid on the patient,
10 -year growth history of the keloid on the patient,
5 -year growth history of the keloid on the patient,
2 -year growth history of the keloid on the patient,
1 -year growth history of the keloid on the patient,
location of the keloid on the patient,
current age of the patient, and
demographic information of the patient,
determine, via a trained AI model, a predicted response of a patient to a steroid dose for the treatment of said keloid; and
output the determined predicted response, wherein the determined predicted response is employed to treatment of the patient by steroid to treat the keloid.

2. The system of claim 1,
wherein the input includes three or more of:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient,
wherein the trained AI model was trained using:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient.

3. The system of claim 1, wherein the input includes:
wherein the input includes three or more of:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient,
current age of the patient, and
demographic information of the patient,
wherein the trained AI model was trained using:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient,
the location of the keloid on the patient,
the current age of the patient, and
demographic information of the patient,

4. The system of claim 1, wherein the trained AI model is a neural network.

5. The system of claim 1, wherein the trained AI model uses at least one of random forest, SVS, XGBoost, logistic regression, gradient boosting, Catboost, bagging, or adaboost.

6. The system of claim 1, wherein the input is provided via a web interface from a web portal.

7. The system of claim 1, wherein the output is provided via a web interface from a web portal.

8. The system of claim 1, wherein the trained AI model was trained without at least one of patient race, keloid trigger information, patient age, keloid onset age, and patient demographic information.

9. A method comprising:
receiving, by a processor, input comprising three or more of:
gender for a patient,
shape of a keloid on the patient,
10 -year growth history of the keloid on the patient,
5 -year growth history of the keloid on the patient,
2 -year growth history of the keloid on the patient,
1 -year growth history of the keloid on the patient,
location of the keloid on the patient,
current age of the patient, and
demographic information of the patient,
determining, by the processor, via a trained AI model, a predicted response of a patient to a steroid dose for the treatment of said keloid; and
outputting, by the processor, the determined predicted response, wherein the determined predicted response is employed to treatment of the patient by steroid to treat the keloid.

10. The method of claim 9,
wherein the input includes three or more of:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient,
wherein the trained AI model was trained using:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient.

11. The method of claim 9, wherein the input includes:
wherein the input includes three or more of:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient,
current age of the patient, and
demographic information of the patient,
wherein the trained AI model was trained using:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient,
the location of the keloid on the patient,
the current age of the patient, and
demographic information of the patient,

12. The method of claim 9, wherein the input is provided via a web interface from a web portal.

13. The method of claim 9, wherein the output is provided via a web interface from a web portal.

14. The method of claim 9, wherein the trained AI model was trained without at least one of patient race, keloid trigger information, patient age, keloid onset age, and patient demographic information.

15. A non-transitory computer-readable medium having instructions stored thereon, wherein execution of the instructions by a processor causes the processor to:
receive input comprising three or more of:
gender for a patient,
shape of a keloid on the patient,
10 -year growth history of the keloid on the patient,
5 -year growth history of the keloid on the patient,
2 -year growth history of the keloid on the patient,
1 -year growth history of the keloid on the patient,
location of the keloid on the patient,
current age of the patient, and
demographic information of the patient,
determine, via a trained AI model, a predicted response of a patient to a steroid dose for the treatment of said keloid; and
output the determined predicted response, wherein the determined predicted response is employed to treatment of the patient by steroid to treat the keloid.

16. The non-transitory computer-readable medium of claim 15,
wherein the input includes three or more of:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient,
wherein the trained AI model was trained using:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient.

17. The non-transitory computer-readable medium of claim 15, wherein the input includes:
wherein the input includes three or more of:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient, and
the location of the keloid on the patient,
current age of the patient, and
demographic information of the patient,
wherein the trained AI model was trained using:
the gender for the patient,
the shape of a keloid on the patient,
the 10-year growth history of the keloid on the patient,
the 5-year growth history of the keloid on the patient,
the 2-year growth history of the keloid on the patient,
the 1-year growth history of the keloid on the patient,
the location of the keloid on the patient,
the current age of the patient, and
demographic information of the patient,

18. The non-transitory computer-readable medium of claim 15, wherein the trained AI model is a neural network.

19. The non-transitory computer-readable medium of claim 15, wherein the trained AI model uses at least one of random forest, SVS, XGBoost, logistic regression, gradient boosting, Catboost, bagging, or adaboost.

20. The non-transitory computer-readable medium of claim 15, wherein the input is provided via a web interface from a web portal, and wherein the output is provided via a web interface from a web portal.
